(19) United States
(12) Patent Application Publication
(10) Pub. No.: US 2023/0320638 A1
(43) Pub. Date: Oct. 12, 2023

(54) Blood Culture Sample Collection Device with Optimized Distal Fluid Path and Pre-Positioned and Sterilized Discard Sample Vacuum Tube

(71) Applicant: Becton, Dickinson and Company, Franklin Lakes, NJ (US)

(72) Inventor: Jonathan Karl Burkholz, Salt Lake City, UT (US)

(21) Appl. No.: 18/132,575

(22) Filed: Apr. 10, 2023

Related U.S. Application Data
(60) Provisional application No. 63/329,645, filed on Apr. 11, 2022.

(51) Int. Cl. A61B 5/15

(52) U.S. Cl. CPC A61B 5/150007; A61M 2039/1077

(57) ABSTRACT

A system for blood sample collection including a container assembly having a cap and defining a reservoir, the cap disposed at a first end of the container assembly, and a fluid access assembly including a housing defining an interior, a fluid access component, a fluid connector component, and an engagement feature, the fluid access component extending from a first end of the housing into the interior of the housing, and the fluid access component defining a lumen. The system also includes a connection portion having a distal end and a proximal end, the connection portion including a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough.

### CROSS-REFERENCE TO RELATED APPLICATION

The present application claims priority to U.S. Provisional Application Ser. No. 63/329,645, entitled “Blood Culture Sample Collection Device with Optimized Distal Fluid Path and Pre-Positioned and Sterilized Discard Sample Vacuum Tube”, filed Apr. 11, 2022, the entire disclosure of which is hereby incorporated by reference in its entirety.

### BACKGROUND OF THE INVENTION

### Field of the Invention

The present disclosure generally relates to systems and methods for collecting a blood sample for blood culture testing from vascular access systems such as, e.g., peripheral intravenous catheters (PIVCs). More particularly, the systems described herein include a blood collection set having an optimized fluid path distal to a luer lock access device (LLAD) for the collection of multiple blood samples.

### Description of Related Art

When collecting a blood specimen from an indwelling vascular access devices, like a peripheral IV catheter (PIVC), a central venous catheter (CVC), or a peripherally inserted central catheter (PICC), the first 1-10 ml of blood is often wasted (i.e., disposed of) to avoid contamination from fluids in the dead space of the vascular access path of such devices. This extra step, while reducing contamination of the blood specimen, may be forgotten or improperly performed, thus compromising the blood specimen.

Blood cultures are often used as a tool to detect the presence of bacteria or fungi in a blood sample of a patient, to identify the type of bacteria or fungi present, and to direct the treatment of the patient. However, accidental contamination of the blood sample is a common problem, causing false positives and often resulting in a patient being prescribed unnecessary treatments such as, e.g., broad spectrum antibiotics. To address this concern, some healthcare providers clean the skin of the patient prior to a blood draw procedure. While this reduces the false positive rate, the rate is still significant (e.g., 3-5%) due to bacteria and/or fungi residing in, e.g., hair follicles. Therefore, some systems also divert a small volume of the initial blood drawn, with the initial (and potentially contaminated) volume being discarded. These systems, however, can be costly and time-consuming. Furthermore, these systems often rely on puncturing a patient's skin to collect the sample, which is uncomfortable for the patient.

Additionally, needle-free blood draw systems, such as PIVO™ from Velano Vascular, Inc., are intended to be used in conjunction with intravenous catheters disposed within the patient. These needle-free blood draw systems may be configured to receive evacuated tubes such that the evacuated tubes, when in fluidic communication with a patient's vasculature system via the needle-free blood draw system, can draw blood into a reservoir of the evacuated tube due to the pressure differential between the reservoir and the patient's vasculature. Since the evacuated tubes are often not provided in a sterile condition, each time an evacuated tube is coupled to the needle-free blood draw system, there may be a risk of contamination. Even when healthcare providers disinfect the surface of a resealable membrane of an evacuated tube (e.g., with an alcohol pad), the contamination risk to the needle-free blood draw system may be too high to use the evacuated tube as a waste tube collector prior to using the needle-free blood draw system to collect a blood sample for blood culturing.

In order to address the potential for sample contamination due to contact with unsterilized evacuated tubes, blood draw systems have been developed such that the evacuated tube configured to receive a discard blood sample is held, via an adapter, in a pre-advanced position within a LLAD, which allows both the evacuated tube and the LLAD to be sterilized together, thereby reducing the number of non-sterile steps and connections during a blood culture collection procedure. Examples of such a system are shown and described in U.S. Application Publication No. 2021/0196167, which is incorporated by reference herein in its entirety. However, while the systems shown and described in U.S. Application Publication No. 2021/0196167 address at least some of the risk of a false positive blood culture sample, they do not typically account for the risk of hemolysis in subsequent blood samples collected after the initial blood culture sample is taken. A common problem with using a vascular access device such as a PIVC to draw blood from a patient is that as blood is drawn into, e.g., a blood collection tube, red blood cells are in a high shear stress state and, thus, are susceptible to hemolysis due to a high pressure differential between the vein and the blood collection tube. Such hemolysis may result in the rejection and discard of a blood sample.

### SUMMARY OF THE INVENTION

In accordance with an aspect of the present disclosure, a system is disclosed, the system including a container assembly including a cap and defining a reservoir, the container assembly having a first end and a second end, the cap disposed at the first end of the container assembly, and a fluid access assembly having a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing. The system also includes a connection portion having a distal end and a proximal end, the connection portion having a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough. The system further includes an adapter including a first engagement feature and a second engagement feature, the first engagement feature of the adapter configured to releasably engage with the cap of the container assembly and the second engagement feature of the adapter configured to releasably engage with the engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

In some embodiments, the fluid path member includes a flexible tube.

In some embodiments, the connection portion includes a compact connector, the compact connector having a proximal connector portion configured to couple the compact connector the fluid connector component, a distal connector portion configured to couple the compact connector to an access port of a vascular access device, and a central portion shaped and configured to reduce hemolysis of a blood sample passing therethrough.

In some embodiments, the connector interface includes one of a threaded luer, a slip luer, a threaded luer lock with collar, a blunt plastic cannula, a male luer, a cannula for PRN access, a needle-free connector, or a needle access cannula.

In some embodiments, the fluid component connector is integrated with the fluid access assembly.

In some embodiments, the fluid component connector is removably coupled to the fluid access assembly.

In some embodiments, the container assembly is configured to be transitioned from the first configuration to a second configuration via translating the container assembly toward the first end of the fluid access assembly such that the cap is disengaged from the first engagement feature of the adapter and the fluid access component pierces a resealable membrane of the cap such that the reservoir of the container assembly is in fluidic communication with the fluid connector component and the connection portion via the lumen of the fluid access component.

In some embodiments, the reservoir of the container assembly is evacuated.

In some embodiments, the engagement feature of the fluid access assembly includes a flange extending outward relative to central axis of the housing of the fluid access assembly.

In some embodiments, the container assembly is a first container assembly, and the system further includes a second container assembly configured to be engaged with the fluid access component after removal of the first container assembly via translating the second container assembly toward the first end of the fluid access assembly such that a resealable membrane of the second container assembly is pierced by the fluid access component and a reservoir of the second container assembly is in fluidic communication with the fluid connector component via the lumen of the fluid access component.

According to another aspect of the present disclosure, a method of using a blood sample collection system is disclosed, the method including providing the blood sample collection system, the system including a container assembly having a cap and defining a reservoir, the container assembly having a first end and a second end, the cap disposed at the first end of the container assembly, a fluid access assembly including a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing, a connection portion having a distal end and a proximal end, the connection portion including a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough, and an adapter including a first engagement feature and a second engagement feature, the first engagement feature of the adapter configured to releasably engage with the cap of the container assembly and the second engagement feature of the adapter configured to releasably engage with the engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component. The method further includes coupling the connector interface of the connection portion to a vascular access device, and translating the container assembly toward the first end of the fluid access assembly and relative to the adapter such that the cap is disengaged from the first engagement feature of the adapter and the fluid access component pierces a resealable membrane of the cap such that the reservoir of the container assembly is in fluidic communication with the fluid connector component via the lumen of the fluid access component. The method also includes decoupling the second engagement feature of the adapter from the engagement feature of the fluid access assembly, and translating the container assembly away from the first end of the fluid access assembly and out of the interior of the fluid access assembly such that the container assembly and the adapter are separated from the fluid access assembly.

In some embodiments, the method further includes sterilizing the blood sample collection system prior to coupling the connector interface of the connection portion to the vascular access device.

In some embodiments, the method includes decoupling the second engagement feature of the adapter from the engagement feature of the fluid access assembly includes at least one of rotating, unlatching, or deforming the adapter relative to the housing of the fluid access assembly.

In some embodiments, the container assembly is a first container assembly, and the method further includes after translating the first container assembly away from the first end of the fluid access assembly and out of the interior of the fluid access assembly, translating a second container assembly toward the first end of the fluid access assembly such that a resealable membrane of the second container assembly is pierced by the fluid access component and a reservoir of the second container assembly is in fluidic communication with the fluid connector component via the lumen of the fluid access component.

In some embodiments, translating the container assembly toward the first end of the fluid access assembly and relative to the adapter such that the fluid access component pierces a resealable membrane of the cap causes the reservoir to draw a blood sample from an indwelling vascular access device fluidically coupled to the connection portion, through the fluid path member, through the fluid access component, and into the reservoir of the container assembly due to the reservoir of the container assembly being evacuated.

In accordance with another aspect of the present disclosure, a system is disclosed, the system including a fluid access assembly having a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing, and the engagement feature of the fluid access assembly disposed on the second end of the housing. The system also includes a container assembly including a cap and an engagement feature, the container assembly defining a reservoir, the container assembly having a first end and a second end, the cap disposed at the first end of the container assembly, the engagement feature of the container assembly configured to releasably engage with engagement feature of the fluid access assembly such that, in a first configuration in which the engagement feature of the adapter is engaged with the engagement feature of the fluid access assembly, the cap is at least partially disposed within the interior of the housing and spaced from the fluid access component. The system further includes a connection portion having a distal end and a proximal end, the connection portion including a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough.

In some embodiments, the fluid path member includes a flexible tube.

In some embodiments, the connection portion includes a compact connector, the compact connector having a proximal connector portion configured to couple the compact connector the fluid connector component, a distal connector portion configured to couple the compact connector to an access port of a vascular access device, and a central portion shaped and configured to reduce hemolysis of a blood sample passing therethrough.

In some embodiments, the container assembly is configured to be transitioned from the first configuration to a second configuration via translating the container assembly toward the first end of the fluid access assembly such that the engagement feature of the container assembly is disengaged from the engagement feature of the fluid access assembly and the fluid access component pierces a resealable membrane of the cap such that the reservoir of the container assembly is in fluidic communication with the fluid connector component and the connection portion via the lumen of the fluid access component.

In some embodiments, the connector interface includes one of a threaded luer, a slip luer, a threaded luer lock with collar, a blunt plastic cannula, a male luer, a cannula for PRN access, a needle-free connector, or a needle access cannula.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a schematic illustration of a blood sample collection system in accordance with an aspect of the present disclosure;

FIG. 2 is a side view of the blood sample collection system of FIG. 1;

FIG. 3 is a perspective view of a compact connector for use with a blood sample collection system in accordance with another aspect of the present disclosure;

FIG. 4A is rear perspective view of the blood sample collection system of FIG. 1 in a first configuration; and

FIG. 4B is a rear perspective view of the blood sample collection system of FIG. 1 in a second configuration.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

While not shown or described herein, it is to be understood that the blood sample collection systems described below may be utilized for blood draw from any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

Embodiments of the present disclosure will primarily be described in the context of blood sample collection systems for use with PIVCs. However, embodiments of the present disclosure equally extend to use with other catheter devices.

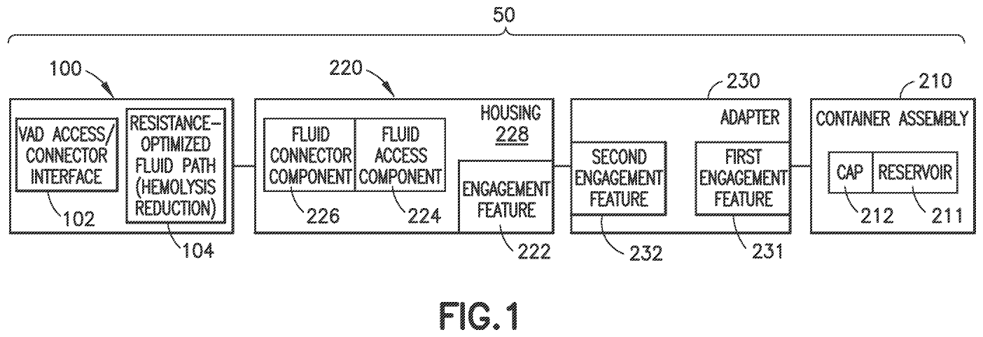

Referring to FIGS. 1, 2, 4A, and 4B, a blood sample collection system 50 in accordance with an aspect of the present disclosure is shown. FIG. 1 is a schematic illustration of the blood sample collection system 50 in accordance with one embodiment. The system 50 includes a container assembly 210, an adapter 230, a fluid access assembly 220, and a connection portion 100. The container assembly 210 may include a cap 212 and can define a reservoir 211. The fluid access assembly 220 may include a housing 228 defining an interior, an engagement feature 222, a fluid access component 224, and a fluid connector component 226.

The adapter 230 may include a first engagement feature 231 and a second engagement feature 232. The first engagement feature 231 of the adapter 230 can be configured to releasably engage with the cap 212 of the container assembly 210, while the second engagement feature 232 of the adapter 230 can be configured to releasably engage with the engagement feature 222 of the fluid access assembly 220. The first engagement feature 231 of the adapter 230 can be or include any suitable engagement mechanism configured to temporarily, or releasably, hold the cap 212 in a position relative to the adapter 230 and to release the cap 212 upon a movement of the cap 212 relative to the first engagement feature 231. The movement can include, for example, a translational movement, a rotational movement, and/or a helical movement. The second engagement feature 232 of the adapter 230 and the engagement feature 222 of the fluid access assembly 220 can be or include any suitable engagement mechanism configured to temporarily, or releasably, engage with each other to temporarily hold the adapter 230 in a position relative to the fluid access assembly 220 or a component of the fluid access assembly 220 (e.g., the housing 228) and to release the adapter 230 from the fluid access assembly 220 (e.g., via a deformation of the adapter 230 resulting from the release of the cap 212 from the adapter 230 and/or via a deformation and/or movement (e.g., rotational, helical, and/or translational) of the second engagement feature 232 and/or the engagement feature 222 relative to the other of the second engagement feature 232 or the engagement feature 222 such that the second engagement feature 232 can be separated from the engagement feature 222).

The container assembly 210 may include a first end and a second end. The cap 212 can be disposed at the first end of the container assembly 210. In some embodiments, the container assembly 210 may include a tube having an open end and a closed end opposite the open end. The cap 212 can be coupled to the open end such that the cap 212 and the tube define the reservoir 211. In some embodiments, the cap 212 may include a resealable membrane. The resealable membrane may be configured such that a fluid access component, such as fluid access component 224, can pierce the resealable membrane to achieve fluidic communication with the reservoir 211. The resealable membrane of the cap 212 may be configured to reseal upon decoupling the fluid access component 224 from the cap 212 such that the reservoir 211 is fluidically isolated from an area external to the container assembly 210. In some embodiments, cap 212 may include ridges and/or one or more flanges disposed on an external surface thereof. The reservoir 211 can be an evacuated reservoir such that, upon the reservoir 211 being placed in fluidic communication with a source of fluid (e.g., via piercing the resealable membrane of the cap 212 with a fluid access component fluidically coupled to a patient's vasculature), fluid (e.g., blood) can be drawn into the reservoir 211 due to a pressure differential between the reservoir 211 and the source of fluid. In some embodiments, the container assembly 210 may be an evacuated tube. In some embodiments, the container assembly 210 may be any suitable standard evacuated tube, such as, e.g., a BD VACUTAINER® from Becton, Dickinson and Co., a Greiner Bio-one® VACUETTE®, etc. The cap 212 may be formed of any appropriate material such as, e.g., rubber. The tube may be formed of, e.g., plastic.

The fluid access assembly 220 may also have a first end and a second end. The fluid access component 224 may be disposed within the interior of the housing 228 and may extend from the first end of the housing 228 into the interior of the housing 228. For example, in some embodiments, the fluid access component 224 may have a first end and a second end opposite the first end. The first end of the fluid access component 224 may be coupled to the first end of the housing 228 and the second end may be disposed in the interior of the housing 228. In some embodiments, the fluid access component 224 may include a needle defining a lumen. In some embodiments, the fluid access assembly 220 may include a flexible needle sheath configured substantially surround and be translated relative to the needle such that a second end of the needle may be selectively exposed.

The fluid connector component 226 may be integrated with or removably coupled to the first end of the housing 228. For example, the housing 228 may define an outlet fluidically coupled to the lumen of the fluid access component 224 to which the fluid connector component 226 can be coupled. In some embodiments, the fluid connector component 226 may include any suitable component configured to couple the housing 228 to patient access tubing. For example, in some embodiments, the fluid connector component 226 may be a luer connector. The luer connector may be in the form of, e.g., a slip luer, a threaded luer, a luer lock with collar, etc. In some embodiments, the fluid connector component 226 can be an outlet of the housing 228 defining a lumen.

Referring still to FIG. 1, the system 50 further includes a connection portion 100, wherein connection portion 100 is configured to reduce hemolysis of the blood samples drawn into the container assembly 210. The connection portion 100 may include a connector interface 102 and a fluid resistance-optimized fluid path member 104. The connector interface 102 may be configured as any appropriate interface capable of coupling the connection portion 100 to an access port of a vascular access device such as, e.g., a PIVC. For example, in some embodiments, the connector interface 102 may be configured as a threaded luer, a slip luer, a threaded luer lock with collar, a blunt plastic cannula (with or without alligator-style connection clips), a male luer (with or without alligator-style connection clips), a cannula for PRN access, a needle-free connector, or a needle access cannula.

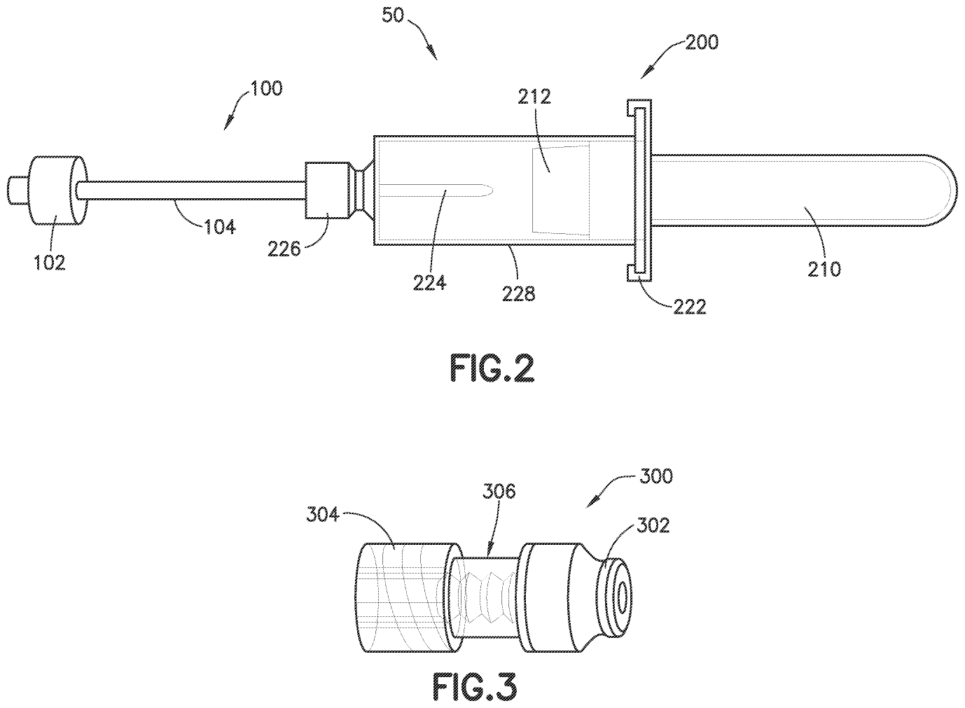

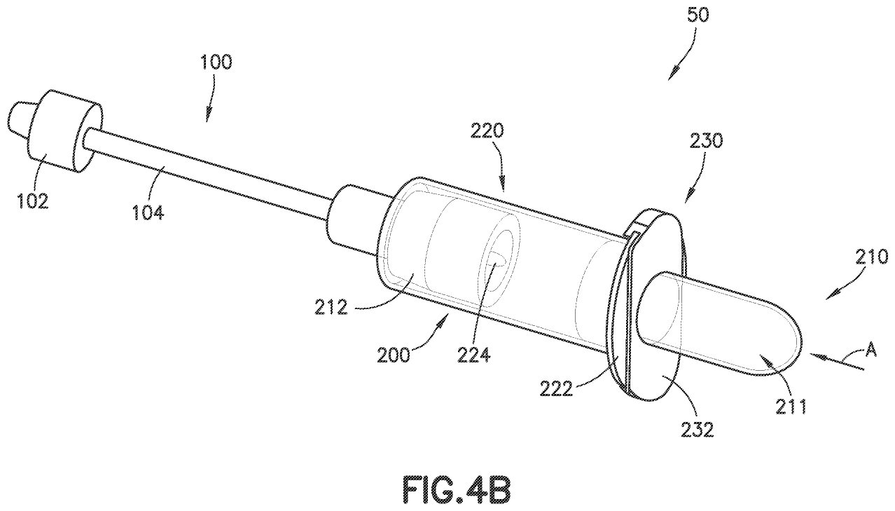

The fluid path member 104 may be formed of any appropriate element capable of providing optimized fluid resistance so as to reduce hemolysis of the blood sample(s) as the blood flows between the connector interface 102 and the fluid connector component 226. In the embodiment shown in FIGS. 2, 4A, and 4B, the fluid path member 104 is formed of flexible tubing. The inner diameter and length of the flexible tubing may be selected so as to limit a maximum blood collection rate which, in turn, may limit a maximum shear stress experienced by the blood cells during blood collection. As noted above, it is shear stress on the blood cells (and particularly on the walls of the blood cells) that is considered a major source of hemolysis and mechanical damage to blood cells. Accordingly, by optimizing fluid path member 104 for fluid resistance, hemolysis of the collected blood sample may be reduced.

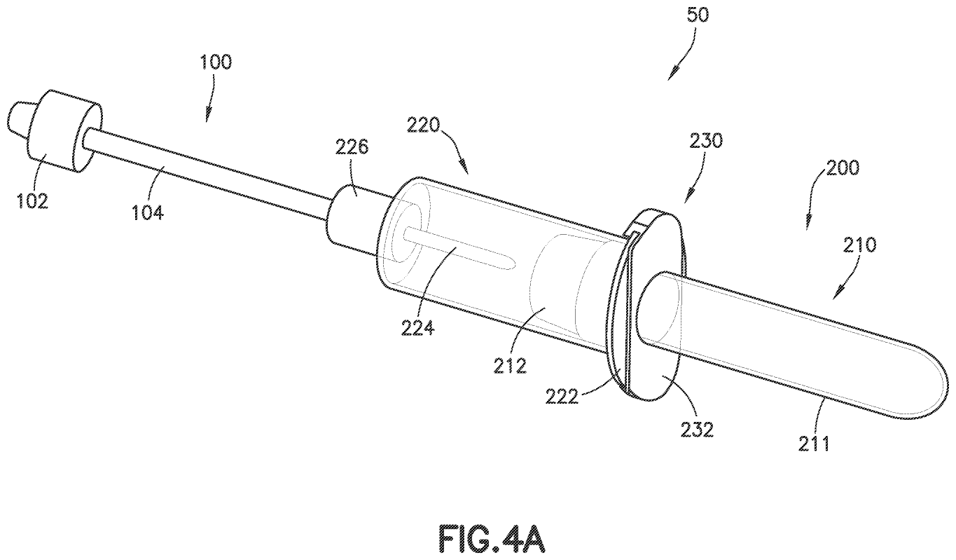

However, while FIGS. 2, 4A, and 4B show the fluid path member 104 formed as flexible tubing, the present disclosure is not limited as such. For example, in some embodiments, fluid path member 104 may be formed as a compact connector, a metal or plastic cannula, or a molded axial fluid path. Referring to FIG. 3, a compact connector 300 in accordance with one aspect of the present disclosure is shown. It is to be understood that compact connector 300 may be utilized in lieu of, e.g., a length of flexible tubing. The compact connector 300 may include a proximal connector portion 302 configured to couple the compact connector 300 to, e.g., the fluid connector component 226 of fluid access assembly 220. A distal connector portion 304 may be provided for coupling the compact connector 300 to, e.g., an access port of a vascular access device. Additionally, a central portion 306 may be provided, with the central portion 306 shaped and configured to increase flow resistance and, thus, reduce hemolysis. Other configurations for fluid path member 104 are also possible. For example, flow path 104 may be configured similar to one or more fluidic resistance-optimized flow paths described in any one of U.S. application Ser. No. 17/146,388, U.S. application Ser. No. 17/401,506, and U.S. application Ser. No. 17/496,858, the disclosures of which are incorporated herein by reference in their entirety.

Referring again to FIGS. 1, 2, 4A, and 4B, the engagement feature 222 of the fluid access assembly 220 may include a flange extending perpendicularly from a central axis of the housing 228. In some embodiments, the flange can be elongated such that the flange extends farther from the central axis of the housing 228 in a first direction than in a second direction. For example, the flange can form an elongated surface of the fluid access assembly 220 disposed in a plane containing the second end of the housing 228.

In some embodiments, the fluid access assembly 220 can be any suitable standard holder, such as, for example, a Greiner Bio-one® VACUETTE® Blood Culture Holder, a JELCO® Saf-T Holder® device sold by Smiths Medical, and/or a BD VACUTAINER® Holder device. In some embodiments, the fluid access assembly connector interface 102 may be configured to couple to and/or otherwise engage an indwelling peripheral intravenous catheter (PIVC). For example, in some embodiments, the connector interface 102 (and, thus, the system 50) may be coupled to any fluid transfer device or portion of a fluid transfer device shown and/or described in U.S. Pat. No. 10,076,272 entitled “Systems and Methods for Phlebotomy through a Peripheral IV Catheter,” filed on Aug. 26, 2014 and/or in U.S. Patent Publication No. 2017/0216564 entitled “Devices and Methods for Fluid Transfer through a Placed Intravenous Catheter,” filed on Feb. 2, 2016, the disclosures of each of which are incorporated by reference herein in their entirety.

The first engagement feature 231 of the adapter 230 may be any suitable feature configured to releasably engage with the cap 212 of the container assembly 210. In some embodiments, the first engagement feature 231 of the adapter 230 may be an inner surface of the adapter 230 that defines a through-hole. The inner surface of the adapter 230 may include a diameter sufficiently small relative to an outermost diameter of the cap 212 such that the inner surface and the cap 212 may be engaged via a friction fit. In some embodiments, the first engagement feature 231 can include a feature corresponding to a feature on the cap 212 such that the adapter 230 and the cap 212 can be releasably engaged.

The second engagement feature 232 of the adapter 230 may be any suitable feature configured to releasably engage with the engagement feature 222 of the fluid access assembly 220. For example, the second engagement feature 232 may include two oppositely-disposed tabs. A latch can be disposed on the end of each tab. Each latch may be shaped and sized to receive a portion of a flange of the engagement feature 222 of the fluid access assembly 220. In some embodiments, the adapter 230 may be rotatable relative to the fluid access assembly 220 such that the second engagement feature 232 (e.g., the latches) can be rotated out of engagement with the engagement feature 222 of the fluid access assembly 220 (e.g., a flange). In some embodiments, the second engagement feature 232 can include a number of arms (e.g., two), each of the arms having a first end coupled to a base of the adapter 230 via a flexible joint and a latch disposed on the opposite end of the arm. In some embodiments, each of the arms may be curved and may form a portion of the outer perimeter of the adaptor 230. Such an adapter can be decoupled from the engagement feature 222 of the fluid access assembly 220 via, for example, rotation and/or deformation (e.g., bending). In some embodiments, the second engagement feature 232 may include a number of tabs including latches configured to be snapped over a flange of the engagement feature 222. For example, the second engagement features 232 may include two or three latching tabs. To separate such an adapter 230 from the housing 228, a user can decouple each latching tab by pulling the latch away from the flange of the engagement feature 222 such that the tab is released from engagement with the flange of the engagement feature 222.

In some embodiments, the fluid access assembly 220 may include a container size adapter such that container assemblies 210 of various sizes and shapes can be used with (e.g., disposed within and stabilized by) the fluid access assembly 220. In some embodiments, the adapter 230 may be configured (e.g., shaped and sized) to engage with an engagement feature of the container size adapter in similar ways as described above with respect to the engagement feature 222 of the fluid access assembly 220. In some embodiments, the adapter 230 may be configured (e.g., shaped and sized) to receive a portion of the container size adapter into the second engagement feature 232 of the adapter 230 when the second engagement feature 232 of the adapter 230 is engaged with the engagement feature 222 of the fluid access assembly 220.

Referring now to FIG. 4A, the system 50 has a first configuration (e.g., an initial configuration) in which the first engagement feature 231 of the adapter 230 is engaged with the cap 212 and the second engagement feature 232 is engaged with the engagement feature 222 of the fluid access assembly 220. When the first engagement feature 231 of the adapter 230 is engaged with the cap 212 and the second engagement feature 232 is engaged with the engagement feature 222 of the fluid access assembly 220, the cap 212 is spaced away from the fluid access component 224 such that the reservoir 211 is fluidically isolated from an external environment of the container assembly 210. In the first configuration, the cap 212 may be at least partially disposed within the adapter 230 and/or the housing 228. For example, a first end of the cap 212 may be disposed within the interior of the housing 228 (either projecting from the adapter 230 or within the adapter 230). In some embodiments, the first end of the cap 212 may be disposed and retained within the adapter 230 but proximal of the housing 228 in the first configuration. In some embodiments, the entire system 50 can be sterilized in the first configuration and packaged for sterile transport to a user (e.g., a healthcare provider).

Next, referring to FIG. 4B, the system 50 also has a second configuration in which the lumen of the fluid access component 224 is in fluidic communication with the reservoir 211. To transition the system 50 from the first configuration to the second configuration, the container assembly 210 may be distally translated in a direction “A” toward the first end of the fluid access assembly 220 such that the cap 212 is engaged with the fluid access component 224 (e.g., the fluid access component 224 pierces the cap 212) and a portion of the fluid access component 224 is disposed within the reservoir 211. For example, in embodiments in which the container assembly 210 is engaged with the first engagement feature 231 via a friction fit, a force may be applied to the container assembly 210 to overcome the force applied by the first engagement feature 231 on the container assembly 210 (e.g., the cap 212) and translate the container assembly 210 into engagement with the fluid access component 224. In the second configuration, when the connector interface 102 is fluidically coupled to a patient's vasculature system, fluid (e.g., blood) can be drawn through the fluid path member 104, though the fluid connector component 226, through the fluid access component 224, and into the reservoir 211 of the container assembly 210.

When sufficient blood has been drawn into the reservoir 211, the system 50 may be transitioned from the second configuration to a third configuration (not shown) in which the container assembly 210 and the adapter 230 are separated from the fluid access assembly 220. For example, the container assembly 210 may be translated relative to the fluid access assembly 220 such that the cap 212 is disposed near the second end of the housing 228. In response to the cap 212 being disengaged from the fluid access component 224, the reservoir 211 may be fluidically isolated from an environment external to the container assembly 210 due to the cap 212 having a resealable membrane. The adapter 230 can then be decoupled from the fluid access assembly 220 via decoupling the second engagement feature 232 from the engagement feature 222 via, e.g., rotating, unsnapping, or deforming one or more portions of the second engagement feature 232 from the engagement feature 222 of the fluid access assembly 220. The adapter 230 and the container assembly 210 may then be optionally discarded, particularly as this initial blood sample may be considered the “discard sample”.

After removing the adapter 230 and the container assembly 210 from the fluid access assembly 220, a second container assembly may be inserted into the interior of the housing and engaged with the fluid access component 224 such that the second container assembly can draw blood into a reservoir of the second container assembly via the fluid connector component 226 and the fluid access component 224. In some embodiments, the second container assembly may include a medium (e.g., a soybean casein digest broth) in the reservoir of the second container configured to be used to perform a blood culture when combined with the patient's blood sample in the reservoir. Any suitable number of container assemblies can be engaged (and subsequently disengaged) with the fluid access component 224 to draw fluid from a patient for various tests. As the system 50 includes the fluid path member 104 coupled to the fluid connector component 226, hemolysis of these subsequent blood collection samples may be reduced. Furthermore, because many of the primary components of system 50 do not need to be detached between blood draws, workflow for the healthcare provider is improved. Additionally, the overall reduction in connections made as compared to conventional blood draw methods may reduce the risk of sample contamination during blood draw. This, coupled with the ability to provide the entirety of system 50 in sterile packaging, reduces the overall risk of contamination of the blood culture samples.

While system 50 described above with respect to FIGS. 1, 2, 4A, and 4B utilize fluid access assembly 220, adapter 230, and container assembly 210 in conjunction with the connection portion 100 for reduced hemolysis of the blood sample, it is to be understood that the present disclosure is not limited as such. That is, the connection portion 100 may be utilized with other blood collection assemblies, including those shown and described in various embodiments of U.S. Application Publication No. 2021/0196167, which is incorporated herein by reference in its entirety.

While several embodiments of blood sample collection system configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. A system, comprising:
a container assembly comprising a cap and defining a reservoir, the container assembly having a first end and a second end, the cap disposed at the first end of the container assembly;
a fluid access assembly comprising a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing;
a connection portion having a distal end and a proximal end, the connection portion comprising a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough; and
an adapter including a first engagement feature and a second engagement feature, the first engagement feature of the adapter configured to releasably engage with the cap of the container assembly and the second engagement feature of the adapter configured to releasably engage with the engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component.

2. The system of claim 1, wherein the fluid path member comprises a flexible tube.

3. The system of claim 1, wherein the connection portion comprises a compact connector, the compact connector comprising a proximal connector portion configured to couple the compact connector the fluid connector component, a distal connector portion configured to couple the compact connector to an access port of a vascular access device, and a central portion shaped and configured to reduce hemolysis of a blood sample passing therethrough.

4. The system of claim 1, wherein the connector interface comprises one of a threaded luer, a slip luer, a threaded luer lock with collar, a blunt plastic cannula, a male luer, a cannula for PRN access, a needle-free connector, or a needle access cannula.

5. The system of claim 1, wherein the fluid component connector is integrated with the fluid access assembly.

6. The system of claim 1, wherein the fluid component connector is removably coupled to the fluid access assembly.

7. The system of claim 1, wherein the container assembly is configured to be transitioned from the first configuration to a second configuration via translating the container assembly toward the first end of the fluid access assembly such that the cap is disengaged from the first engagement feature of the adapter and the fluid access component pierces a resealable membrane of the cap such that the reservoir of the container assembly is in fluidic communication with the fluid connector component and the connection portion via the lumen of the fluid access component.

8. The system of claim 1, wherein the reservoir of the container assembly is evacuated.

9. The system of claim 1, wherein the engagement feature of the fluid access assembly includes a flange extending outward relative to central axis of the housing of the fluid access assembly.

10. The system of claim 1, wherein the container assembly is a first container assembly, and further comprising a second container assembly configured to be engaged with the fluid access component after removal of the first container assembly via translating the second container assembly toward the first end of the fluid access assembly such that a resealable membrane of the second container assembly is pierced by the fluid access component and a reservoir of the second container assembly is in fluidic communication with the fluid connector component via the lumen of the fluid access component.

11. A method of using a blood sample collection system, comprising:
providing the blood sample collection system, the system comprising:
a container assembly comprising a cap and defining a reservoir, the container assembly having a first end and a second end, the cap disposed at the first end of the container assembly,
a fluid access assembly comprising a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing,
a connection portion having a distal end and a proximal end, the connection portion comprising a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough, and
an adapter including a first engagement feature and a second engagement feature, the first engagement feature of the adapter configured to releasably engage with the cap of the container assembly and the second engagement feature of the adapter configured to releasably engage with the engagement feature of the fluid access assembly such that, in a first configuration in which the first engagement feature of the adapter is engaged with the cap and the second engagement feature of the adapter is engaged with the engagement feature of the fluid access assembly, the cap of the container assembly is at least partially disposed within the interior of the housing and spaced from the fluid access component;
coupling the connector interface of the connection portion to a vascular access device;
translating the container assembly toward the first end of the fluid access assembly and relative to the adapter such that the cap is disengaged from the first engagement feature of the adapter and the fluid access component pierces a resealable membrane of the cap such that the reservoir of the container assembly is in fluidic communication with the fluid connector component via the lumen of the fluid access component;
decoupling the second engagement feature of the adapter from the engagement feature of the fluid access assembly; and
translating the container assembly away from the first end of the fluid access assembly and out of the interior of the fluid access assembly such that the container assembly and the adapter are separated from the fluid access assembly.

12. The method of claim 11, further comprising sterilizing the blood sample collection system prior to coupling the connector interface of the connection portion to the vascular access device.

13. The method of claim 11, wherein decoupling the second engagement feature of the adapter from the engagement feature of the fluid access assembly includes at least one of rotating, unlatching, or deforming the adapter relative to the housing of the fluid access assembly.

14. The method of claim 11, wherein the container assembly is a first container assembly, the method further comprising:
after translating the first container assembly away from the first end of the fluid access assembly and out of the interior of the fluid access assembly, translating a second container assembly toward the first end of the fluid access assembly such that a resealable membrane of the second container assembly is pierced by the fluid access component and a reservoir of the second container assembly is in fluidic communication with the fluid connector component via the lumen of the fluid access component.

15. The method of claim 11, wherein translating the container assembly toward the first end of the fluid access assembly and relative to the adapter such that the fluid access component pierces a resealable membrane of the cap causes the reservoir to draw a blood sample from an indwelling vascular access device fluidically coupled to the connection portion, through the fluid path member, through the fluid access component, and into the reservoir of the container assembly due to the reservoir of the container assembly being evacuated.

16. A system, comprising:
a fluid access assembly including a housing defining an interior, a fluid access component, a fluid connector component, and an engagement feature, the housing having a first end and a second end, the fluid access component extending from the first end of the housing into the interior of the housing, the fluid access component defining a lumen, the fluid connector component disposed on the first end of the housing, and the engagement feature of the fluid access assembly disposed on the second end of the housing;
a container assembly including a cap and an engagement feature, the container assembly defining a reservoir, the container assembly having a first end and a second end, the cap disposed at the first end of the container assembly, the engagement feature of the container assembly configured to releasably engage with engagement feature of the fluid access assembly such that, in a first configuration in which the engagement feature of the adapter is engaged with the engagement feature of the fluid access assembly, the cap is at least partially disposed within the interior of the housing and spaced from the fluid access component; and
a connection portion having a distal end and a proximal end, the connection portion comprising a connector interface disposed at the distal end and a fluid path member fluidly coupled to the connector interface and configured to be coupled to the fluid connector component of the fluid access assembly, wherein the fluid path member is configured to reduce hemolysis of a blood sample passing therethrough.

17. The system of claim 16, wherein the fluid path member comprises a flexible tube.

18. The system of claim 16, wherein the connection portion comprises a compact connector, the compact connector comprising a proximal connector portion configured to couple the compact connector the fluid connector component, a distal connector portion configured to couple the compact connector to an access port of a vascular access device, and a central portion shaped and configured to reduce hemolysis of a blood sample passing therethrough.

19. The system of claim 16, wherein the container assembly is configured to be transitioned from the first configuration to a second configuration via translating the container assembly toward the first end of the fluid access assembly such that the engagement feature of the container assembly is disengaged from the engagement feature of the fluid access assembly and the fluid access component pierces a resealable membrane of the cap such that the reservoir of the container assembly is in fluidic communication with the fluid connector component and the connection portion via the lumen of the fluid access component.

20. The system of claim 16, wherein the connector interface comprises one of a threaded luer, a slip luer, a threaded luer lock with collar, a blunt plastic cannula, a male luer, a cannula for PRN access, a needle-free connector, or a needle access cannula.
